(19) United States
(12) Patent Application Publication
(10) Pub. No.: US 2007/0259396 A1
(43) Pub. Date: Nov. 8, 2007

(54) PROCESS FOR PRODUCING PLANT-ORIGIN ANTIBACTERIAL SUBSTANCE

(76) Inventor: Takuo Sakai, Osaka (JP)

Correspondence Address: BIRCH STEWART KOLASCH & BIRCH, PO BOX 747, FALLS CHURCH, VA, 22040-0747, US

(21) Appl. No.: 11/744,517

(22) Filed: May 4, 2007

Related U.S. Application Data
(63) Continuation-in-part of application No. 10/069,182, filed on May 22, 2002, now abandoned.

(30) Foreign Application Priority Data
Jun. 23, 2000 (JP) 2000-189614

(51) Int. Cl. C12P 1/00; A23L 3/34

(52) U.S. Cl. 435/41; 426/532

(57) ABSTRACT

A process for producing an antibacterial substance which comprises disintegrating at least a part of tissue of the plant with an enzyme capable of acting on protopectin to release and recover the antibacterial substance therefrom, while a pectin substance is removed; and antibacterial or bacteriostatic compositions containing the antibacterial substance thus obtained as the active ingredient. By using the above process and compositions, the proliferation of spore-forming bacteria and germination of spores from spore-forming bacteria and koji mold can be efficiently inhibited.

### CROSS-REFERENCE TO RELATED APPLICATIONS

The present application is a continuation-in-part of U.S. application Ser. No. 10/069,182 filed May 22, 2002, which claims priority on PCT International Application No. PCT/JP01/04929 filed Jun. 11, 2001, which in turn claims priority on Japanese patent Application No. JP 2000-189614 filed Jun. 23, 2000.

### TECHNICAL FIELD

The present invention relates to a process of producing an antibacterial substance derived from a plant which includes disintegrating at least a part of tissue of the plant and releasing the antibacterial substance therefrom, and bactericidal or bacteriostatic compositions containing as an active ingredient the antibacterial substance obtained by the process.

### BACKGROUND ART

Microorganisms, especially bacteria which form spores (spore forming bacteria), often contaminate food and do great economic damage. Processed food and others are inviting targets for contamination by spore forming bacteria. If even one spore remains after sterilization of food, bacteria easily proliferate to damage the quality of the food significantly.

Among such spore forming bacteria, there exist those producing poisonous matters (cereus toxin, botulinum toxin) which may bring death to human beings such as aerobic Bacillus cereus and anaerobic Clostridium botulinum.

Accordingly, the prevention and extermination of spore forming bacteria is an important problem to solve in the food industry. However, spore forming bacteria always exist in agricultural products which are raw materials, and often contaminate processed food.

Bacterial spores can survive even under conditions which generally kill bacteria such as high temperature, presence of organic solvents, dryness and the like. For example, the bacterial spores do not die out when left in boiling water for 20 minutes, and they do not die out under a condition of 2% or less moisture.

Accordingly, even food having been sterilized at high temperatures is often contaminated with spore forming bacteria. Furthermore, since spores are smaller than cells, they cannot be removed by bacteria elimination such as microfiltration in many cases. This is also a reason for difficult control of spore forming bacteria.

For the above reasons, various kinds of substances inhibiting proliferation of spore forming bacteria have been studied, but no substances have been found that are food-hygienically safe and effective.

Tissue of plants of the higher orders is constructed of aggregates of cells, and pectins play an important part in this construction. In plant tissue, pectins bind via rhamnogalactan to cellulose or hemicellulose which constitutes cell walls, and then form so-called middle lamellae of multi-layered structure by chelate bond via a bivalent metal such as calcium to bond cells and form tissue.

Insoluble pectins in this form are referred to as protopectins, and enzymes which have the activity of acting on protopectins and freeing pectin substances are generally referred to as protopectinases (Fermentation and Industry: 37, 928-938, 1978; Agric. Biol. Chem., 52, 1091-1093, 1988; Agric. Biol. Chem., 53, 1213-1223, 1989; Agric. Biol. Chem., 54, 879-889, 1990; Eur. J. Biochem., 226, 285-291, 1994; Biosci. Biotech. Biochem., 58, 353-358, 1994). If protopectinases are allowed to act on plant tissue, cells are isolated to be single cells while water-soluble pectin substances are released.

### DISCLOSURE OF INVENTION

Noting that dead plants are easily decomposed by bacteria while living plants are not contaminated by ordinary bacteria except for plant pathogenic microbes, the inventor has studied on this biological mechanism, finally to find out that substances solubilized from middle lamellae together with pectins when protopectinases act on plants have the nature of inhibiting proliferation of bacteria. Thus the present invention has been accomplished.

According to the present invention, there are provided a process of producing an antibacterial substance from a plant which includes disintegrating at least a part of tissue of the plant with an enzyme capable of acting on protopectin to release and recover the antibacterial substance therefrom, while a pectin substance is removed, and a bactericidal or bacteriostatic composition containing as an active ingredient the antibacterial substance obtained by the process.

### BRIEF DESCRIPTION OF DRAWINGS

FIG. 1 shows results of a proliferation inhibition test of a supernatant of onion which is single-celled by treatment with protopectinase-S against Bacillus subtilis. A halo observed around a hole for placing a liquid to be tested indicates the inhibition of proliferation;

FIG. 2 shows an example of a proliferation inhibition test of a plant extract treated with protopectinase-S against Bacillus subtilis;

FIG. 3 shows the result of the test for inhibition of germination of spores of Bacillus subtilis in Example 9; and

FIG. 4 shows charts of HPLC obtained in Example 9.

### BEST MODE FOR CARRYING OUT THE INVENTION

The plant used for the process of the present invention is not particularly limited, and may be a dicotyledon or a monocotyledon. As examples of dicotyledons, may be included Ranales (water lily, peony), Piperales (Houttuynia cordata, pepper), Cucurbitales (pumpkin, dishcloth gourd, bitter gourd), Opuntiales (cactus), Rosales (cherry, pear, bean, strawberry, loquat, arrowroot), Rutales (mango, orange, lemon), Plantaginales (plantain), Umbelliflorae (dropwort, honewort, carrot), Asterales (Japanese butterbur, dandilions, edible burdock, garland chrysanthemum, mugwort), Polemoniales (potato, capsicum, tobacco, sesame, sweet potato), Rhoeadales (poppy, Chinese cabbage, cabbage, turnip, brassica), Malvales (cotton, gumbo) and the like. As examples of monocotyledons, may be mentioned Liliales (lily, garlic, onion, aloe), Arales (colocasis), Amaryllidales (welsh onion, Chinese chive), Iridales (iris, sweet flag), Dioscoreales (Chinese yam, yam), Agavales (agave), Orchidales (orchid), Graminales (rice, barley, the Korean lawn grass, cane, corn) and the like.

Any parts of these plants such as terrestrial stems, subterranean stems, leaves, roots, flowers, fruits, seeds, buds and sprouts may be used. For example, terrestrial stems and leaves of cabbage, garland chrysanthemum, mugwort, dandelion and dropwort, subterranean stems of potato and onion, roots of sweet potato and carrot, flowers of cotton, fruits of pumpkin and sprout of bean may be used.

The plants may be used as they are or after they are cut or ground into an appropriate size. However, in order to disintegrate plant tissue sufficiently, the plants may preferably be chipped into an appropriate size, for example, a square of about 0.5 to 1 cm. If the plants have sizes larger than this range, the plants may be ground by combining a mechanical means such as a Waring blender.

The plants release antibacterial substances existing in intercellular spaces by being treated with an enzyme capable of separating plant cells into individual cells under stirring.

The enzyme may be any enzyme that can act on a protopectin to release a pectin substance. The enzyme may be used singly, or two or more kinds of enzymes may be combined for use.

Examples of enzymes that may be included protopectinases, polymethyl galacturonases, polygalacturonase, arabinases and rhamnogalacturonases. More particularly, protopectinase-F, protopectinase-S, protopectinase-L, protopectinase-T, protopectinase-C, protopectinase-N and polymethyl galacturonase SX1 may preferably be used.

Treatment conditions may be selected as appropriate depending upon the kind of the enzyme and the kind and weight of the plant. In the case where a protopectinase or polymethyl galacturonase is used as an enzyme, the enzyme is added in an amount of 1,500 to 15,000 units, preferably 1,500 to 10,000 units, more preferably 2,000 to 5,000 units with respect to 10 g (wet weight) of the plant. The treatment is carried out at 30 to 40° C., ideally at about 37° C. for one to ten hours.

The plant can be treated with the enzyme in acetate buffer, phosphate buffer, Tris HCl buffer, physiologic saline or water, within the range of pH 2.0 to 10.0, preferably at pH 7.0, and especially it is preferable to carry out the treatment in the acetate buffer, phosphate buffer and Tris HCl buffer. If the weight of the plant is equal, the finer the plant is cut, the sooner the antibacterial substance is released.

The liquid obtained after the above-described treatment with the enzyme is subjected to a step of removing a pectin substance. The step of removing a pectin substance can be carried out by a conventional method. Such conventional method includes, for example, a separation of pectin substances by using anion exchange resin, a separation of pectin substances by ethanol precipitation.

The liquid obtained after the removal of a pectin substance may be used as it is. Alternatively, the liquid may be made into a supernatant partially purified by conventional filtration and centrifugation or into an isolated liquid or solid product by further filtration and purification, if desired.

For purification, various means usually used such as cation exchanger resins, ammonium sulfate precipitation using ammonium sulfate of 80% concentration, gel filtration using dextran gel, polyacrylamide gel or agarose gel and the like may be used singly or in optional combination, though the antibacterial substances contained in the liquids treated with the enzyme do not necessarily have similar structures depending upon the plants from which the antibacterial substances are derived.

For example, in the case where a cation exchanger resin is used for a column method, CM-cellulose, Amberlite, CM-Sephadex and CM-TOYOPEARL may be used as resins. In the case of a liquid containing the antibacterial substance obtained by treating sweet potato with protopectinase-S, the supernatant is passed through a column fed with CM-TOYOPEARL buffered with phosphate buffer (pH 7.0) in order that the antibacterial substance is adsorbed, and further a 0.6 M aqueous solution of sodium chloride is passed through the column to elute and separate the antibacterial substance.

According to the tests conducted by the present inventor, the antibacterial substances obtained by the above-described method have antibacterial activity against spore forming bacteria typified by bacilli and clostridia and also have antibacterial activity against koji mold of Aspergilli. From this, it is considered that the antibacterial substances suppress the proliferation of spore forming bacteria and koji mold by the action of inhibiting germination of spores.

Accordingly, the antibacterial substances of the present invention can be used for bactericidal or bacteriostatic compositions in various preparations such as aqueous liquid preparations, aerosols, solid preparations (powder, granule) and the like. As ideal carriers for these preparations include carriers known in the field of art such as water, starch, wheat flour, sucrose, glucose, lactose, dextrin and the like. Further, pectin, albumin, arabinan and others may be optionally added to the compositions.

The antibacterial substances of the present invention can be used for preventing food from decaying by being mixed with or sprayed on various kinds of food such as bread, noodles, candies, cookies, soft drinks, nourishing drinks and jellies in process or at the final step in the form of the above-mentioned preparations optionally in combination with other bactericidal agents or bacteriostatic agents. Also the antibacterial substances can be used as products of other industrial fields in the fields of feed, quasi drugs and the like.

Thus, since the antibacterial substances of the present invention can be produced from raw material plants which have been eaten as vegetables, fruit vegetables, medical herbs and others since ancient times, it is specially mentioned that the antibacterial substances can be utilized as means for preventing and exterminating spore forming bacteria by being added to food without causing adverse effects on human beings.

Further, since any parts of plants can be used as materials in the production process and as materials for the antibacterial agents according to the present invention, agricultural products substandard for market as well as cooking scraps are effectively used. Therefore, the invention will make extremely great contributions to society, including the prevention of environmental contamination due to waste food and the creation of new merchandizes, and its economical effect will be unexpectedly great.

The present invention is now explained with reference to examples, which should not be construed to limit the scope of the invention.

### EXAMPLE 1

An onion (a subterranean stem), 10 g (wet weight), was chopped into 0.5 to 1 cm squares and suspended in 10 mL of 100 mM acetate buffer (pH 7). The enzymes shown in Table 1, 4,000 units, were added to the resulting suspension, followed by stirring at 37° C. for 5 hours (the activity of the enzynes was determined in accordance with Sakai's method in Methods in Enzymology, Academic Press, vol. 161, pp. 335 to 350). As a result, the tissue of the onion was disintegrated and a liquid was produced which contained single cells and intracellular substances.

This liquid was filtered with a 20-mesh cloth filter of nylon to obtain a filtrate, which was centrifuged at 2,000 g for 5 minutes to completely remove insoluble substances. The anti-bacterial activity of the resulting supernatant was determined by a modification of the cylinder plate method (Antibiotics Handbook, edited by Kazumaro ICHINO and Hiroshi MUROYA, published by Sangyo Tosho, page 181; Summary of Antibiotics, written by Nobuo TANAKA and Shoshiro NAKAMURA, Tokyo University Publishing Association, page 24). More particularly, the supernatant, 50 μL, was fed in 6 mm diameter holes made on a flat plate of a potato dextrose agar medium (Nissui Seiyaku Kabushiki Kaisha) inoculated with spores of Bacillus subtilis, allowed to stand at 15° C. for three hours, and then kept at 37° C. for 24 hours to allow Bacillus subtilis to proliferate. The diameter of proliferation inhibition circles formed around the holes was measured to determine the antibacterial activity against Bacillus subtilis.

The antibacterial activity was determined to be one unit when the diameter of the proliferation inhibition circle minus the diameter of the hole, i.e., 6 mm, was 1 cm. The antibacterial activity of the enzymes was calculated with use of the enzymes alone and the buffer alone as controls.

The results of this experiment are shown in Table 1.

TABLE 1

| | Antibacterial |
|---|---|
| Enzymes Used | Activity (unit/mL) |
| Protopectinase - F ¹⁾ | 43.0 |
| Protopectinase - S ²⁾ | 44.2 |
| Polymethyl galacturonase - SX1 ²⁾ | 41.0 |
| Protopectinase - L ¹⁾ | 44.3 |
| Protopectinase - T ³⁾ | 21.3 |
| Protopectinase - N ⁴⁾ | 45.1 |
| Controls | 0 |

¹⁾ T. Sakai, Methods in Enzymology, Vol. 161, 335-350, 1988, Academic Press
²⁾ T. Sakai et al., FEBS Letters, Vol. 414, 439-443, 1997
³⁾ M. Sakamoto et al., Eur. J. Biochem., Vol. 226, 285-291, 1994
⁴⁾ T. Sakai et al., Adv. Appl. Microbiol., Vol. 36, 213-294, 1993

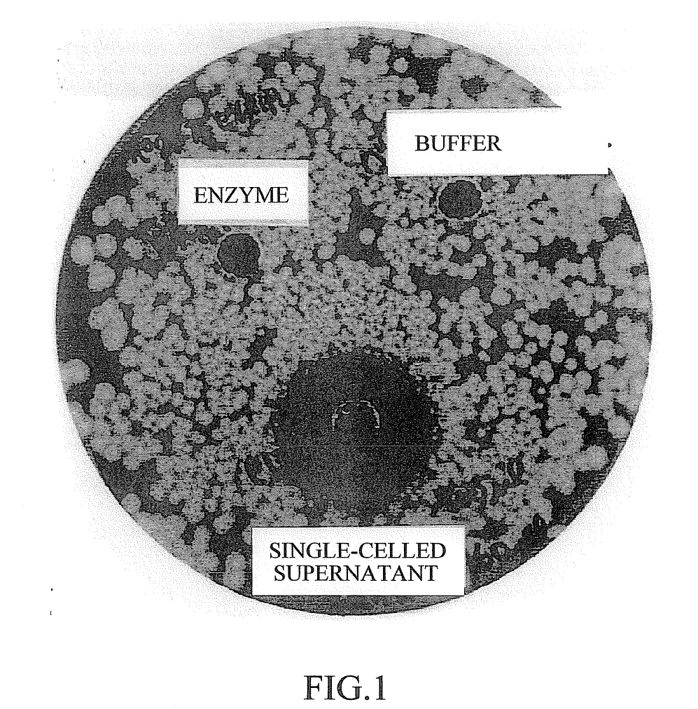

With the controls, Bacillus subtilis grew immediately around the holes, while bacteria did not grow around the holes into which the enzyme-treated supernatants were fed. It was confirmed that the supernatants had remarkable antibacterial activities. Therefore, it is clear that this method can produce a substance inhibiting the growth of Bacillus subtilis from onion (See FIG. 1).

### EXAMPLE 2

Various species of plants weighting 3 g chopped into pieces of 0.5 to 1 cm square were suspended in 10 mL of 100 mM acetate buffer pH 7 containing protopectinase-S (500 units). The resulting suspensions were stirred at 37° C. for an hour and then were centrifuged in the manner described in Example 1. The antibacterial activity of the supernatants against Bacillus subtilis was determined in the same manner as described in Example 1. The antibacterial activity was recognized with all the plants as shown in Table 2.

TABLE 2

| | Antibacterial |
|---|---|
| Plants used (parts) | Activity (unit/mL) |
| Sweet potato (root) | 47.0 |
| Pumpkin (fruit) | 49.3 |
| Cabbage (terrestrial stem and leaves) | 61.2 |
| Garland chrysanthemum | 34.0 |
| (terrestrial stem and leaves) | |
| Carrot (root) | 60.2 |
| Potato (subterranean stem) | 61.2 |
| Onion (subterranean stem) | 44.2 |
| Mugwort (terrestrial stem and leaves) | 34.0 |
| Dandelion | 18.1 |
| (terrestrial stem and leaves) | |
| Dropwort (terrestrial stem and leaves) | 22.5 |
| Cotton (flowers) | 11.5 |
| Control (enzyme alone) | 0 |

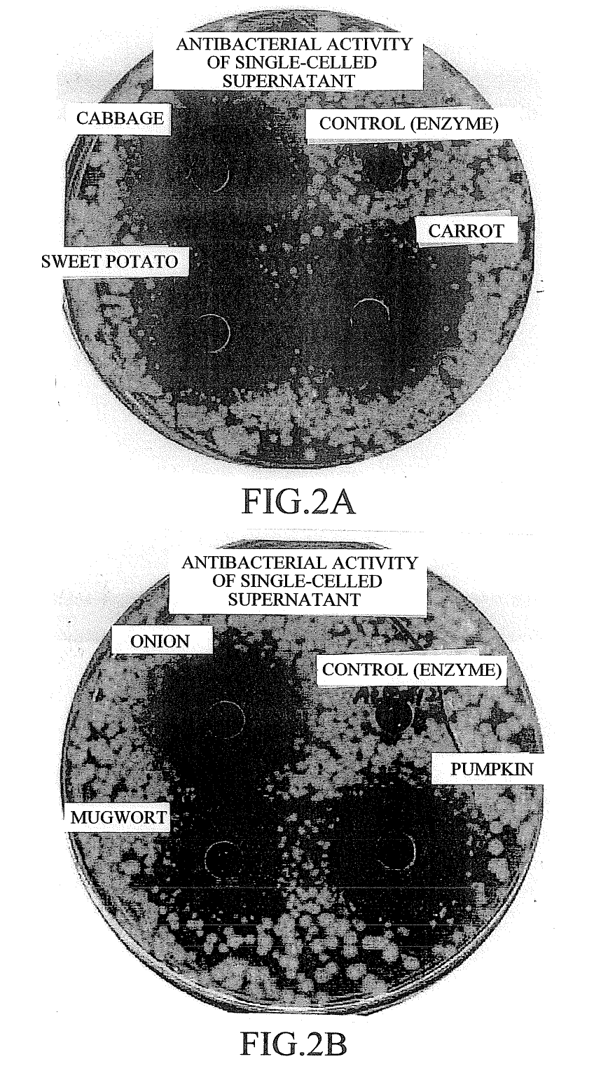

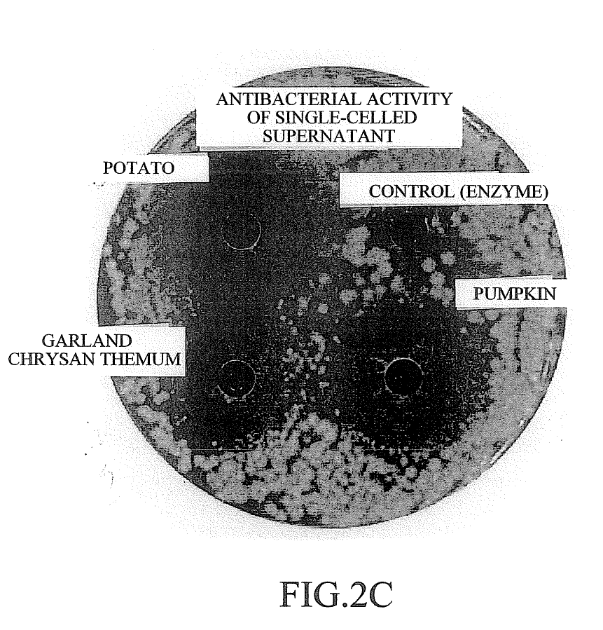

These results clearly show that antibacterial substances exist widely in plants and that plants can be used as materials for antibacterial substances regardless of species and parts (see FIG. 2)

### EXAMPLE 3

Various species of plants (parts used were the same as in Example 2) were chopped into pieces of 1 to 2 cm square and suspended in 100 mM acetate buffer pH 7, and then were completely ground at 5° C. using a Waring blender. The supernatants obtained by removing insoluble substances by centrifugation were tested for their antibacterial activity against bacillus subtilis in the same manner as in Example 1.

The results show that the plants had the antibacterial activity as shown in Table 3.

TABLE 3

| Plants used | Antibacterial Activity (unit/mL) |
|---|---|
| Sweet potato | 7.0 |
| Pumpkin | 5.3 |
| Cabbage | 11.2 |
| Garland chrysanthemum | 13.0 |
| Carrot | 6.5 |
| Potato | 7.3 |
| Onion | 34.2 |
| Mugwort | 24.2 |
| Control (enzyme alone) | 0 |

Thus, it was proved that not only liquids obtained by disintegrating plant tissue with enzymes but also liquids of ground plants obtained by mechanical technique contained antibacterial substances.

### EXAMPLE 4

The activity of liquids containing antibacterial substances prepared from a pumpkin (fruit) and a sweet potato (root) according to the process of Example 1 using protopectinase-S were determined with regard to the microorganisms shown in Table 4.

TABLE 4

| | Antibacterial activity (Activity | |
|---|---|---|
| | of sweet potato extract against | |
| | Bacillus subtilis | |
| | is assumed to be 100) | |
| Microorganisms | Sweet potato ex. | Pumpkin ex. |
| Bacillus subtilis IFO 3134 | 100 | 290 |
| Bacillus cereus IFO 3001 | 113 | 283 |
| Bacillus alvei IFO 14175 | 110 | 300 |
| Bacillus sphaericus IFO 3528 | 98 | 267 |
| Bacillus pumilus IFO 3030 | 132 | 301 |
| Bacillus megaterium AKU 212 | 121 | 305 |
| Bacillus amyloliquefacienece | 30 | 51 |
| IFO 14141 | | |
| Bacillus circulans IFO 33239 | 32 | 48 |
| Bacillus coagulans IFO 12583 | 30 | 56 |
| Bacillus firms IFO 3330 | 38 | 55 |
| Bacillus licheniformis IFO | 28 | 42 |
| 14206 | | |
| Bacillus macerance IFO 3490 | 42 | 68 |
| Bacillus natto IFO 3013 | 56 | 80 |
| Clostridium acetobutylicum | 81 | 230 |
| ATCC 3625 | | |
| Aspergillus awamori IFO 4033 | 11 | 25 |

Table 4 clearly shows that the antibacterial substances produced by the process of the present invention inhibit the growth not only of Bacillus subtilis but also of bacteria belonging to Bacillus family and bacteria belonging to Aspergillus family, and therefore can be used as antibacterial agents.

### EXAMPLE 5

Sweet potato, 900 g, chopped into pieces of about 1 cm square were suspended in a 100 mM phosphate buffer pH 7. Protopectinase-S, 200,000 units, was added to the resulting suspension and the resulting mixture was allowed to react at 37° C. for five hours with stirring. The tissue of the sweet potato was completely disintegrated to produce single cells and intercellular liquid.

The single cells in the treated liquid were removed according to Example 1 to give a supernatant containing 60,000 units of an antibacterial substance.

### EXAMPLE 6

Bean sprouts of black matpe, 1 kg, were cut to a length of about 5 mm and suspended in a 100 mM Tris-HCl buffer (pH 7.0) containing 2% of sodium chloride. Protopectinase (Pectinase-GODO manufactured by Godo Shusei K.K.), 5 g (115,000 I.U.) was added to the resulting suspension and the resulting mixture was allowed to react at 37° C. for six hours with stirring. After reaction, cellular residues were filtered off with a nylon mesh and the filtrate was centrifuged at 21,000 g for 20 minutes. The resulting supernatant was observed to have an antibacterial activity of 10 unit/mL.

Pectin, porcine serum albumin, dextrin, arabinan and soluble starch (potato) were added to the supernatant so that the final concentration thereof was 0.5%. The resulting mixtures were allowed to stand at 10° C. for 10 days. Thereafter, the antibacterial activity in the mixtures was determined to give the results shown in Table 5.

Liquids containing the antibacterial substance which were stable were thus obtained by adding various substances.

TABLE 5

| Additive | Antibacterial activity (unit/mL) |
|---|---|
| No additive (control) | 8.0 |
| Pectin | 10.0 |
| Porcine serum albumin | 10.0 |
| Dextrin | 9.5 |
| Arabinan | 10.0 |
| Soluble starch | 10.0 |

### EXAMPLE 7

Dextrin was added to and dissolved in 10 mL of the supernatant obtained in Example 6 so that the final concentration was 5%. This solution was lyophilized to produce a powder-form antibacterial composition.

This composition was dissolved in a 2% saline and the antibacterial activity was determined to be 5.5 unit/g. It was confirmed that the antibacterial activity was maintained in a solid state.

### EXAMPLE 8

Soluble starch (potato) was added to 10 mL of the supernatant obtained in Example 6 so that the final concentration was 5%. The resulting mixture was stirred well and then dried under vacuum while maintaining the temperature of 10° C. or lower.

The thus obtained solid powder was dissolved in a 2% saline, and a precipitate generated was removed. Then the antibacterial activity in the supernatant was determined. Five units of the antibacterial activity was observed per gram of the solid powder.

Thus the antibacterial composition was produced in a solid state using soluble starch.

### EXAMPLE 9

Aloe (Aloe arborescens, 400 g) was soaked into 1.5 L of water at 80° C. After removal of water, Protopectinase-S as shown in Table 1 (10⁵ units/g, 40 g) which was dissolved into 4 L of water was added, followed by stirring at 37° C. for 20 hours. The resulting solution was then centrifuged (8,000 rpm, 20 min), and the supernatant was concentrated under reduced pressure to 4-fold (1 L).

Isopropanol (3 L) was added to the concentrated supernatant (1 L) and the mixture was centrifuged (8,000 rpm, 20 min). The supernatant was concentrated to dryness under reduced pressure. The obtained solid was dissolved into distilled water to adjust the volume to 250 mL. The obtained solution was further purified with Sep-Pak® Plus C18 Environmental Cartridges (Waters) as follows.

The Sep-Pak Cartridge was washed with 10 mL of acetonitrile and equilibrated with 10 mL of distilled water. The above solution (1 mL) was loaded on the Cartridge and eluted with 10 mL of distilled water. The eluent was concentrated to 1 mL to obtain a crude Aloe extract.

A mini-column (BIO-RAD, 8×40 mm) was filled with an anion-exchange resin (DIAION® SA11A; Mitsubishi Chemical Corporation). The column was washed with 150 mM acetate buffer (pH 7) so that the resin was substituted into acetate form. The above crude Aloe extract was loaded on the column, followed by elution with 150 mM acetate buffer (pH 7). By this step, pectin contained in the crude Aloe extract was absorbed on the resin and removed. The flow-through of the column was collected, concentrated under reduced pressure, and acetate was removed. The product was dissolved into 1 mL of water. Thus obtained solution was used in the following experiments as the antibacterial substance solution.

(1) Test for Inhibition of Germination of Spores (Antibacterial Test)

GYP liquid medium (0.85 mL, pH was adjusted to 7.0 with phosphate buffer) was added with 0.15 mL of the antibacterial substance solution and 0.1 mL of spore-containing liquid (5×10⁴ spores/mL, Bacillus subtilis, Eiken Chemical Co., Ltd.) and incubated at 37° C. for 150 minutes. The reacted medium (0.1 mL) was spread on a GYP-agar plate and incubated overnight at 37° C. Photographs of plates were taken.

The similar tests were carried out with using a solution of pectin (from lemon; 20 mg/mL-water) or sterilized water (negative control) instead of the above antibacterial solution.

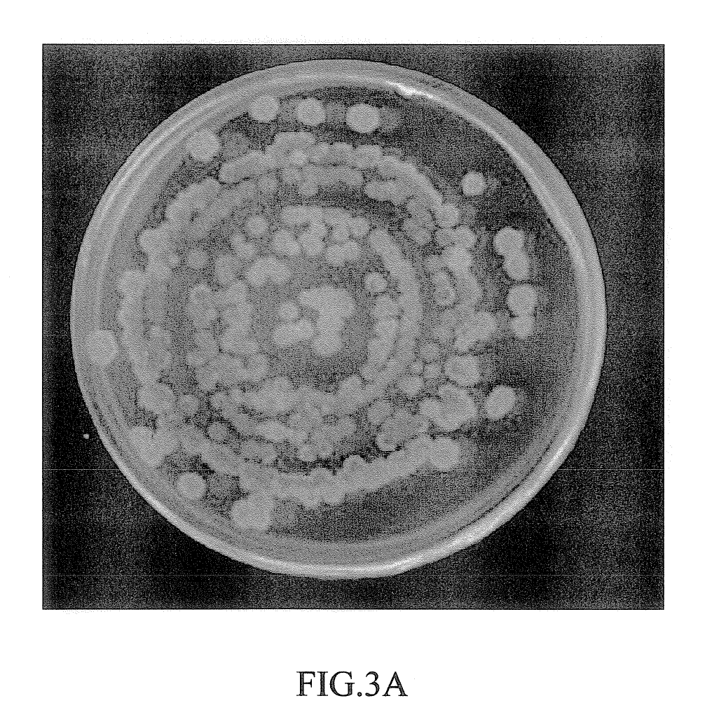

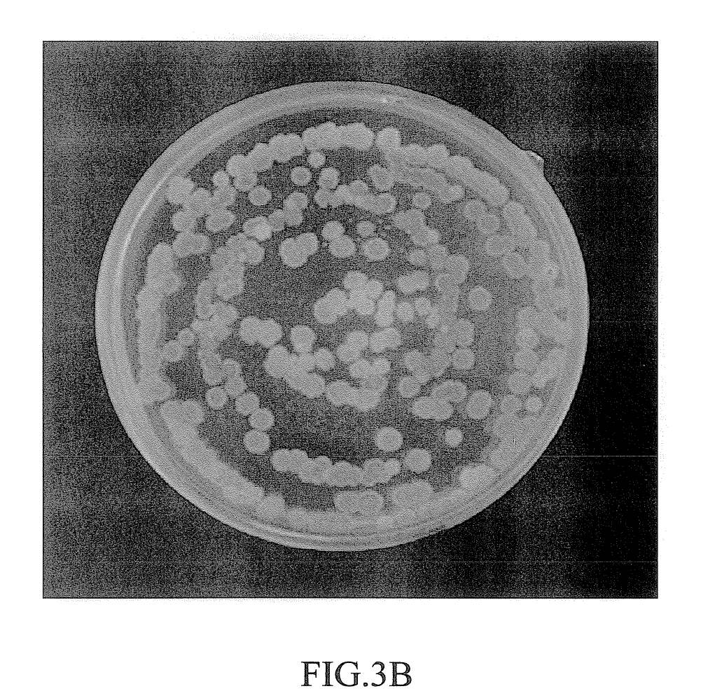

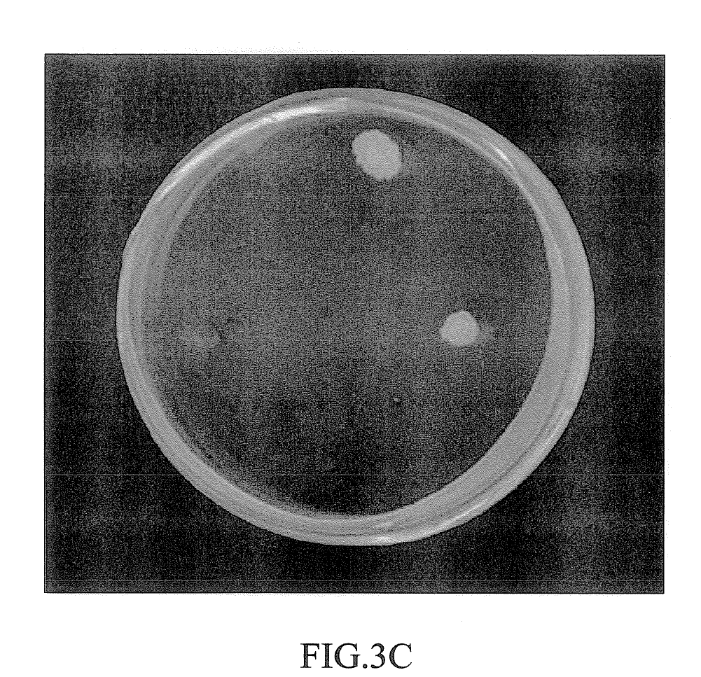

Results are shown in FIG. 3. FIG. 3 (a) shows the plate added with sterilized water; (b) shows the plate added with the solution of pectin; and (c) shows the plate added with the above antibacterial substance solution.

From the results shown in FIG. 3, it is recognized that the antibacterial substance solution has an inhibitory effect of germination of spores of spore forming bacteria, while the solution of pectin does not have such inhibitory effect.

(2) Analysis of Antibacterial Substance and Pectin by High Performance Liquid Chromatography (HPLC)

HPLC analyses were conducted on the above crude Aloe extract obtained after the reaction with Protopectinase-S, the above antibacterial substance solution obtained after removal of pectin by anion-exchange resin, and the solution of pectin (from lemon; 20 mg/mL-water).

The condition for HPLC was as follows:
Sample volume: 0.2 mL;
Column: COSMOSIL 5C₁₈-AR-300, 10×250 mm (Waters);
Column temperature: 40° C.;
Flow rate: 1.0 mL/min
Elution: solution A: 0.2% formic acid in distilled water, solution B: 0.065% formic acid in acetonitrile;
Gradient: from 95% solution A to 40% solution A in 40 minutes;
Detector: HITACHI L-4000 UV Detector.

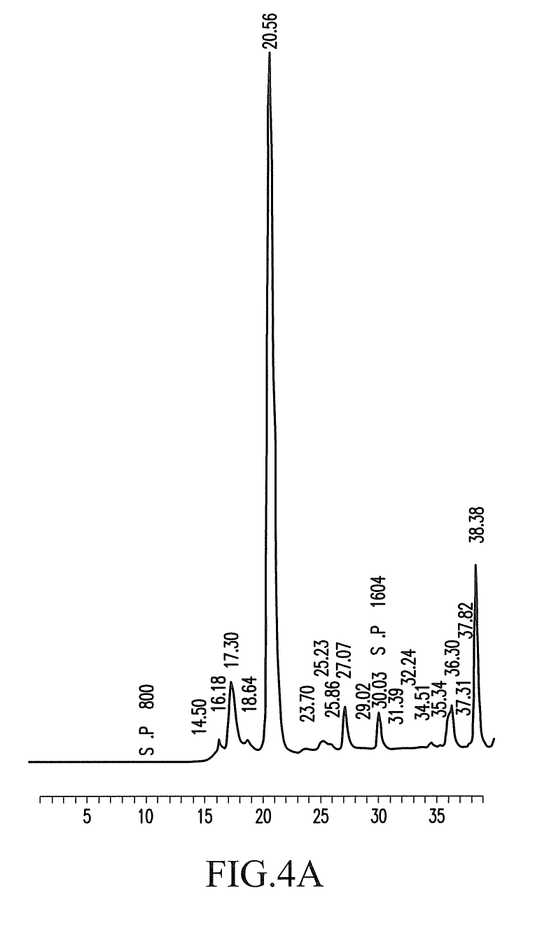

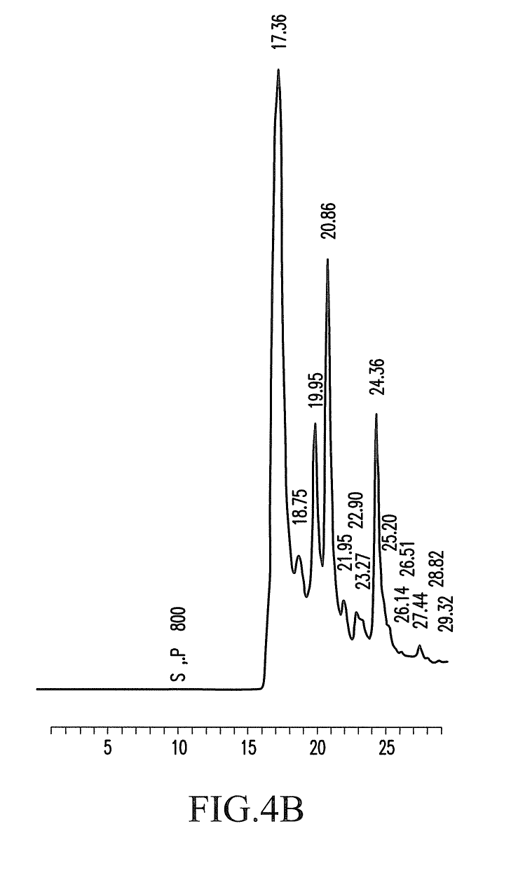

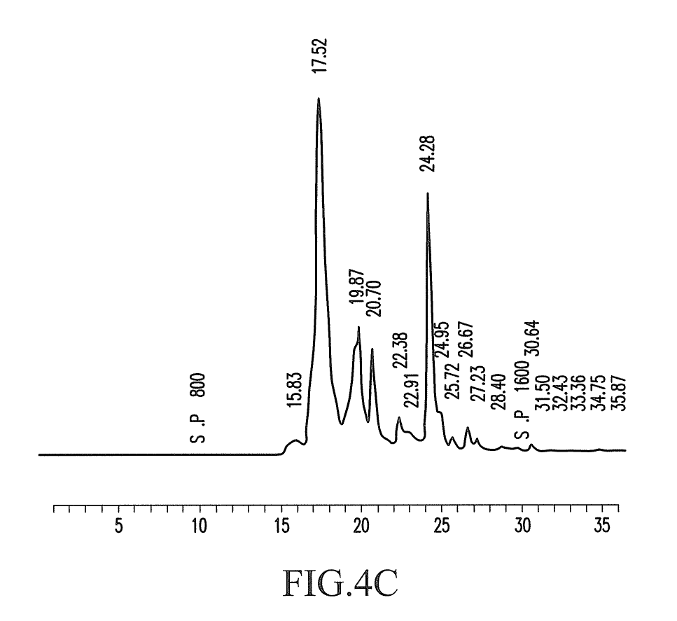

Results are shown in FIG. 4. FIG. 4 (a) shows a chart obtained from the solution of pectin; (b) shows a chart obtained from the crude Aloe extract after the reaction with Protopectinase-S; and (c) shows a chart obtained from the above antibacterial substance solution obtained after removal of pectin.

From the results shown in FIG. 4, it is recognized that pectin is eluted at around 20 minutes in the above HPLC condition and that the treatment by the anion-exchange resin could remove most of pectin from the crude Aloe extract. Further, it can be said that the inhibitory effect of germination of spores demonstrated in the test (1) is effected by the antibacterial substance and that pectin itself does not have any effect for inhibition of germination of spores.

According to the present invention, there are provided a process of producing an antibacterial substance derived from a plant, the process including releasing the antibacterial substance by disintegrating at least part of tissue of the plant and a bactericidal or bacteriostatic composition containing, as an effective ingredient, the antibacterial substance obtained by this process.

The antibacterial substance can prevent contamination by spore forming bacteria which has been difficult so far. Also since a wide variety of plants can be used as materials for the antibacterial substance regardless of their species or parts, safe antibacterial substances can be obtained economically.

1. A process of producing an antibacterial substance derived from a plant, which comprises disintegrating at least a part of tissue of the plant with an enzyme capable of acting on protopectin to release and recover the antibacterial substance therefrom, while a pectin substance is removed,
the plant being optionally cut or ground to an appropriate size.

2. The process according to claim 1, wherein the enzyme is at least one selected from the group consisting of protopectinases, polymethyl galacturonases, polygalacturonases, arabinases and rhamnogalacturonases.

3. The process according to claim 1 or 2, wherein the enzyme is protopectinase-F, -S, -L, -T, -C or -N or polymethyl galacturonase-SX1.

4. The process according to claim 1, wherein the enzyme is used within a range from pH 2.0 to 10.0 at a temperature of 30 to 40° C.

5. The process according to claim 1, wherein the plant is selected from the group consisting of cabbage, garland chrysanthemum, mugwort, dandelion, dropwort, potato, onion, sweet potato, carrot, cotton and pumpkin.

6. The process according to claim 1, wherein the antibacterial substance inhibits germination of spores of spore-forming bacteria and koji mold.

7. A bactericidal or bacteriostatic composition containing the antibacterial substance obtained by the process according to claim 1 as an effective ingredient.

8. A food containing the composition according to claim 7.

9. The food according to claim 8, which is bread, noodle, candies, cookies, soft drink, nourishing drink or jelly.
